(19) United States
(12) Patent Application Publication
(10) Pub. No.: US 2025/0332405 A1
(43) Pub. Date: Oct. 30, 2025

(54) DEVICES, SYSTEMS, AND METHODS FOR RECORDING ELECTROPHYSIOLOGICAL SIGNALS OR FOR STIMULATING TISSUE

(71) Applicants: Jerel MUELLER, Dover, DE (US); Jiashu LI, Dover, DE (US)

(72) Inventors: Jerel MUELLER, Dover, DE (US); Jiashu LI, Dover, DE (US)

(21) Appl. No.: 19/186,475

(22) Filed: Apr. 22, 2025

Related U.S. Application Data
(60) Provisional application No. 63/640,783, filed on Apr. 30, 2024.

(51) Int. Cl. A61N 1/05; A61N 1/36

(52) U.S. Cl. CPC A61N 1/05; A61N 1/3605

(57) ABSTRACT

Disclosed herein are devices, systems, and methods for recording bio-signals and/or stimulating tissue. In one aspect, an implantable medical device is disclosed comprising a conductive frame, one or more insulating layers disposed on the conductive frame, and at least one metallic component affixed to the one or more insulating layers. A first conductive element can be electrically coupled to at least part of the conductive frame and a signal analyzer and a second conductive element can be electrically coupled to the at least one metallic component and the signal analyzer. The signal analyzer can be configured to determine a voltage signal using the conductive frame as the reference electrode and the at least one metallic component as the active electrode. Alternatively, the signal analyzer can also determine a voltage signal using the conductive frame as the active electrode and the at least one metallic component as the reference electrode.

### CROSS-REFERENCE TO RELATED APPLICATION

This application claims the benefit of U.S. Provisional Application No. 63/640,783, filed Apr. 30, 2024, the content of which is incorporated herein by reference in its entirety.

### FIELD OF TECHNOLOGY

The present disclosure relates generally to the field of implantable medical devices, and, more specifically, to devices, systems, and methods for recording electrophysiological signals or for stimulating tissue.

### BACKGROUND

Millions of people around the world suffer from various neuromuscular or neurological disorders or diseases where control of limbs is severely impaired or limited. For these people, the ability to restore lost control, at even a rudimentary level, could lead to a greatly improved quality of life. One option for restoring function to such individuals is a brain computer interface (BCI) system. Such systems often include one or more implantable devices for recording a subject's neural activity and, in some cases, the same implantable devices or additional implantable devices can be used to stimulate the subject's neural tissue.

Neural recordings from cortical neurons are oftentimes dependent on separating the recorded extracellular potential of neurons near the recording electrode from ambient sources of noise. For example, such noise can include motion artifacts, 60-Hz noise, instrumentation noise, thermal noise, or other biological sources of noise. The usefulness of a recording signal depends on minimizing sources of noise while maximizing signal strength. Currently, one method for doing this is using an appropriate reference electrode.

Selecting such a reference electrode is a balance of trade-offs. For example, while a large-sized reference electrode placed more remotely from the recording electrode(s) can boost neuronal signal amplitudes, such a reference electrode can pick up additional noise along its conduction pathway or new noise around the vicinity of the reference electrode. Also, there are physical limitations on the size of the reference electrode when it comes to implantation sites within cranial vessels, the subarachnoid space, or cranial extravascular locations. Moreover, a reference electrode that is placed too close to the recording electrode(s) may inadvertently pick up the target neural signal.

Therefore, a solution is needed for addressing the above-mentioned limitations. Such devices or systems should be able to robustly separate the recorded extracellular potential of neurons from ambient sources of noise and be implantable within different cranial locations (e.g., cranial vessels, the subarachnoid space, or cranial extravascular locations). Such a solution should also not be overly complicated.

### SUMMARY

Disclosed herein are devices, systems, and methods for recording bio-signals and/or stimulating tissue within a subject. For example, an implantable medical device is disclosed comprising a conductive frame and one or more insulating layers disposed on at least part of the conductive frame. The implantable medical device can further comprise at least one metallic component affixed to the one or more insulating layers, a first conductive element electrically coupled to at least part of the conductive frame, and a second conductive element electrically coupled to the at least one metallic component.

The first conductive element and the second conductive element can be configured to be electrically coupled to a signal analyzer configured to determine a voltage signal within a subject using the conductive frame as a reference electrode and the at least one metallic component as an active electrode.

A Iso disclosed is a method of analyzing a signal comprising determining, using one or more processors of a signal analyzer, a voltage signal within a subject using a conductive frame of an implantable medical device as a reference electrode and at least one metallic component of the implantable medical device as an active electrode. The implantable medical device can comprise one or more insulating layers disposed on at least part of the conductive frame, a first conductive element electrically connecting at least part of the conductive frame to the signal analyzer, and a second conductive element electrically connecting the at least one metallic component to the signal analyzer. The at least one metallic component can be affixed to the one or more insulating layers.

The method can further comprise transmitting the voltage signal from the signal analyzer to an extracorporeal computing device to calculate a power spectral density (PSD) or to perform additional signal processing based on the voltage signal.

In some embodiments, an entirety of the conductive frame can serve as the reference electrode. For example, the entirety of the conductive frame can have a total conductive frame surface area and the at least one metallic component can have a total component electrode surface area. A ratio of the total conductive frame surface area to the total component electrode surface area can be between about 3:1 and 30:1.

In other embodiments, a portion of the conductive frame can serve as the reference electrode. For example, the portion of the conductive frame can be between about 25% to about 95% of the entirety of the conductive frame by surface area. Also, for example, the portion of the conductive frame can be electrically insulated from another portion of the conductive frame such that the conductive frame is divided into multiple electrically conductive regions.

In some embodiments, the conductive frame can be substantially tubular-shaped. In other embodiments, the conductive frame can be substantially planar or flattened.

The conductive frame can comprise a plurality of struts connected by crosslinks. Each of the struts can be a wire formed into at least one of a wave-like or undulating shape, a zig-zag shape, a straight line, or a combination thereof. The at least one metallic component can be positioned at a linkage point or meeting point between adjacent struts.

The conductive frame can be made of a shape memory metallic alloy. For example, the conductive frame can be made of a nickel-titanium alloy (e.g., Nitinol).

The first conductive element and the second conductive element can be conductive traces embedded within or extending through the one or more insulating layers.

In some embodiments, at least one of the insulating layers can be made of yttria stabilized zirconia or silicon dioxide.

The at least one metallic component can be made of a noble metal or noble metal alloy. For example, the at least one metallic component can be made of at least one of gold, titanium, and platinum.

In some embodiments, the signal analyzer can be part of a telemetry unit. For example, the signal analyzer, or the telemetry unit, can be configured to be implanted within the body of a subject.

In other embodiments, the signal analyzer can be an extracorporeal device.

The voltage signal can be at least one of a field potential, an event potential, and a neuronal action potential.

A Iso disclosed is an implantable medical device comprising a conductive frame and one or more insulating layers disposed on at least part of the conductive frame. The implantable medical device can further comprise at least one metallic component affixed to the one or more insulating layers, a first conductive element electrically coupled to at least part of the conductive frame, and a second conductive element electrically coupled to the at least one metallic component.

The first conductive element and the second conductive element can be configured to be electrically coupled to a signal analyzer configured to determine a voltage signal within a subject using the conductive frame as an active electrode and the at least one metallic component as a reference electrode.

A Iso disclosed is a method of analyzing a signal comprising determining, using one or more processors of a signal analyzer, a voltage signal within a subject using a conductive frame of an implantable medical device as an active electrode and at least one metallic component of the implantable medical device as a reference electrode. The implantable medical device can comprise one or more insulating layers disposed on at least part of the conductive frame, a first conductive element electrically connecting at least part of the conductive frame to the signal analyzer, and a second conductive element electrically connecting the at least one metallic component to the signal analyzer. The at least one metallic component can be affixed to the one or more insulating layers.

The method can further comprise transmitting the voltage signal from the signal analyzer to an extracorporeal computing device to perform signal processing of the voltage signal. In additional embodiments, the computing device can calculate a power spectral density (PSD) based in part on the voltage signal.

In some embodiments, an entirety of the conductive frame can serve as the active electrode. For example, the entirety of the conductive frame can have a total conductive frame surface area and the at least one metallic component can have a total component electrode surface area. A ratio of the total conductive frame surface area to the total component electrode surface area can be between about 3:1 and 30:1.

In other embodiments, a portion of the conductive frame can serve as the active electrode. For example, the portion of the conductive frame can be between about 25% to about 95% of the entirety of the conductive frame by surface area. Also, for example, the portion of the conductive frame can be electrically insulated from another portion of the conductive frame such that the conductive frame is divided into multiple electrically conductive regions.

In some embodiments, the conductive frame can be substantially tubular-shaped. In other embodiments, the conductive frame can be substantially planar or flattened.

The conductive frame can comprise a plurality of struts connected by crosslinks. Each of the struts can be a wire formed into at least one of a wave-like or undulating shape, a zig-zag shape, a straight line, or a combination thereof. The at least one metallic component can be positioned at a linkage point or meeting point between adjacent struts.

The conductive frame can be made of a shape memory metallic alloy. For example, the conductive frame can be made of a nickel-titanium alloy (e.g., Nitinol).

The first conductive element and the second conductive element can be conductive traces embedded within or extending through the one or more insulating layers.

In some embodiments, at least one of the insulating layers can be made of yttria stabilized zirconia or silicon dioxide.

The at least one metallic component can be made of a noble metal or noble metal alloy. For example, the at least one metallic component can be made of at least one of gold, titanium, and platinum.

In some embodiments, the signal analyzer can be part of a telemetry unit. For example, the signal analyzer, or the telemetry unit, can be configured to be implanted within the body of a subject.

In other embodiments, the signal analyzer can be an extracorporeal device.

The voltage signal can be at least one of a field potential, an event potential, and a neuronal action potential.

A Iso disclosed is an implantable stimulation system. The implantable stimulation system can comprise a conductive frame and a pulse generator electrically coupled to the implantable conductive frame. The conductive frame can be configured to be implanted near an intracorporeal target within a subject. The pulse generator can be configured to generate an electrical impulse that is transmissible to the implantable conductive frame to stimulate the intracorporeal target.

Moreover, disclosed is a method of stimulating an intracorporeal target. The method can comprise generating an electrical impulse using a pulse generator electrically coupled to a conductive frame implanted near an intracorporeal target. The pulse generator can be electrically coupled to a conductive frame implanted near an intracorporeal target. The conductive frame can stimulate the intracorporeal target in response to the electrical impulse generated by the pulse generator.

In some embodiments, the conductive frame can be configured to be implanted within the brain of the subject (e.g., within cortical vessels). The conductive fame can also be configured to be implanted at locations along the surface of the brain, at locations exterior to brain vessels, or within the dura mater of the subject.

In some embodiments, an entirety of the conductive frame can be used to stimulate the intracorporeal target.

In other embodiments, a portion of the conductive frame can be used to stimulate the intracorporeal target. For example, the portion of the conductive frame can be between about 25% to about 95% of an entirety of the conductive frame by surface area.

In certain embodiments, the implantable medical device can be a stent electrode array and the conductive frame can be a frame of the stent electrode array.

In some embodiments, the conductive frame can be substantially tubular-shaped. In other embodiments, the conductive frame can be substantially planar or flattened.

The conductive frame can comprise a plurality of struts connected by crosslinks. For example, each of the struts can be a wire formed into at least one of a wave-like or undulating shape, a zig-zag shape, and a straight line.

In some embodiments, the conductive frame can be made of a shape memory metallic alloy. For example, the conductive frame can be made of a nickel-titanium alloy (e.g., Nitinol). The pulse generator can be part of an implantable telemetry unit.

In some embodiments, generating the electrical impulse can further comprise generating the electrical impulse by increasing a current amplitude of the electrical impulse from 0 mA to up to 10 mA in 0.1 mA steps. Moreover, generating the electrical impulse can further comprise increasing a voltage of the electrical impulse from 0 V to up to 10 V in 0.25 V steps. In addition, a pulse width of the electrical impulse generated can be configured to be between about 25 uS to about 600 uS. Furthermore, a frequency of the electrical impulse generated can be configured to be between 0.5 Hz and 10,000 Hz.

### BRIEF DESCRIPTION OF THE DRAWINGS

FIG. 1A illustrates one embodiment of an implantable medical device for recording bio-signals and/or stimulating tissue within a subject.

FIG. 1B illustrates one embodiment of a system for recording bio-signals and/or stimulating tissue comprising the implantable medical device.

FIG. 2A is a schematic representation of a cross-section of part of the implantable medical device.

FIG. 2B illustrates a close-up partially exploded view of an electrode mounting site of the implantable medical device.

FIG. 3A illustrates another embodiment of the implantable medical device comprising a plurality of electrodes affixed to a conductive frame.

FIGS. 3B-3E illustrate additional embodiments of the implantable medical device comprising conductive frames of various shapes and configurations.

FIGS. 3F-31 illustrate alternative embodiments of the implantable medical device comprising flattened or planar-shaped conductive frames

FIG. 4 is a graph comparing the use of the conductive frame as a reference electrode against using a conductive case of the signal analyzer as the reference electrode.

FIG. 5 are graphs comparing the use of the conductive frame as a reference electrode and the use of various metallic components as reference electrodes.

FIG. 6 illustrates certain veins and sinuses of a subject that can serve as possible implantation sites for the medical device disclosed herein.

FIG. 7 illustrates a method of analyzing a bio-signal obtained from within a body of a subject.

FIG. 8 illustrates another method of analyzing a bio-signal obtained from within the body of a subject FIG. 9 illustrates a method of stimulating an intracorporeal target.

### DETAILED DESCRIPTION

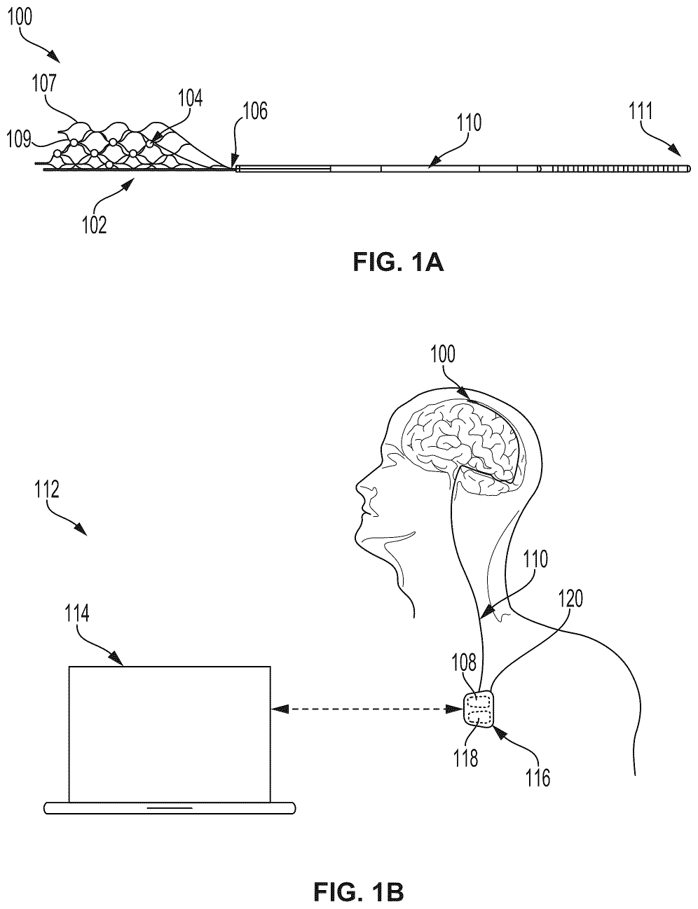

FIG. 1A illustrates one embodiment of an implantable medical device 100 for recording bio-signals and/or stimulating tissue within a subject. For example, the implantable medical device 100 can be configured to record neural signals and/or stimulate neural tissue within the brain of a subject or at locations along the surface of the brain, at locations exterior to brain vessels, or at locations within the dura mater of the subject.

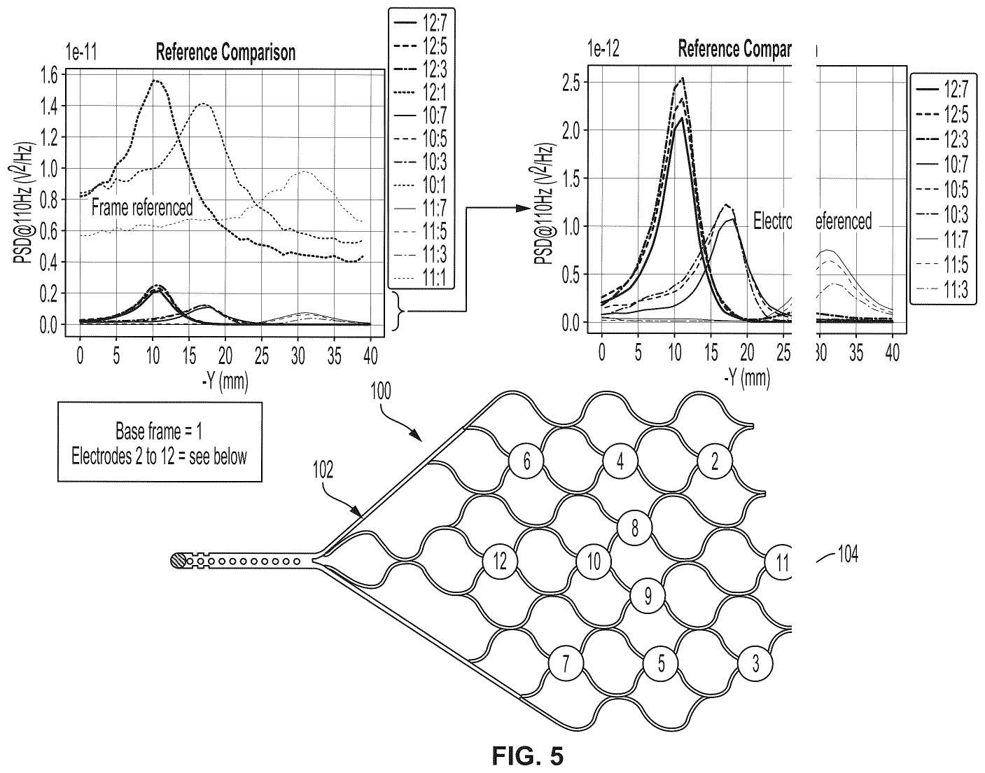

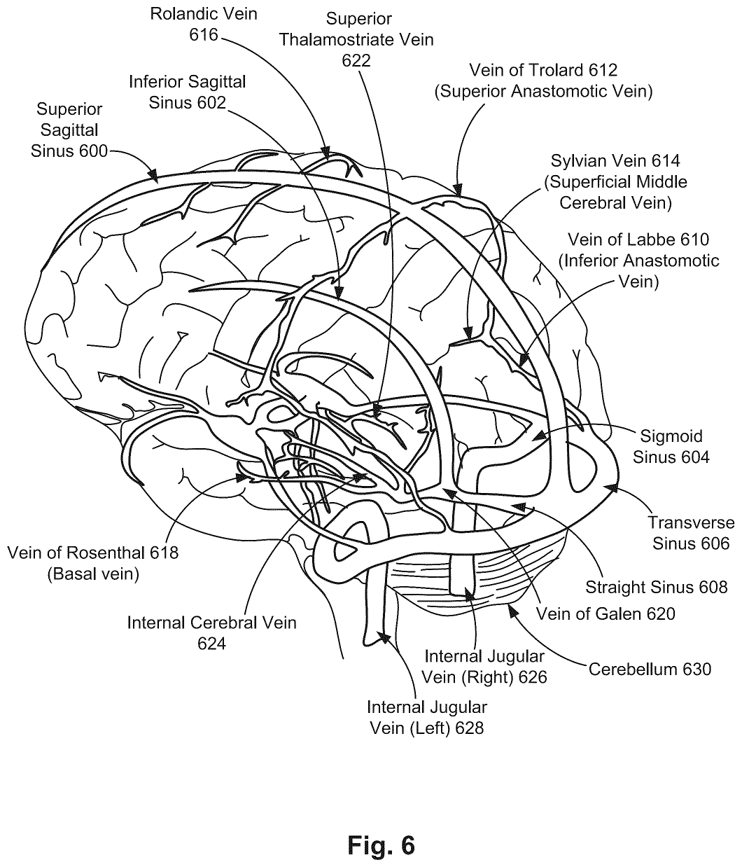

In some embodiments, the implantable medical device 100 can be configured to be implanted within a cranial or neural/cerebral vessel (e.g., a neural blood vessel or sinus), the subarachnoid space, or a cranial extravascular location (see FIG. 6 for additional implantation sites).

In certain embodiments, the implantable medical device 100 can be used to conduct vagal nerve recordings or stimulate the vagal nerve. In other embodiments, the implantable medical device 100 can be used to conduct renal nerve recordings or stimulate the renal nerve.

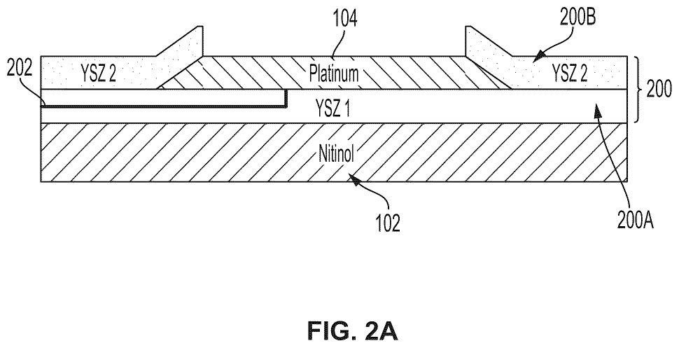

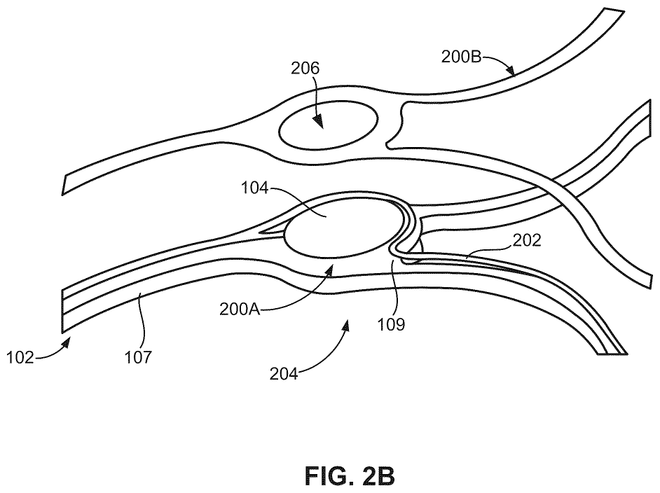

The implantable medical device 100 can comprise a conductive frame 102 and one or more insulating layers 200 (see, e.g., FIGS. 2A and 2B) disposed on at least part of the conductive frame 102. In some embodiments, the one or more insulating layers 200 can cover a radially outward-facing surface or upper/top surface of the conductive frame 102. In these embodiments, the one or more insulating layers 200 do not cover an inner surface, a radially inward-facing surface, or a bottom surface of the conductive frame 102 (which are left exposed by the one or more insulating layers 200). In certain embodiments, the one or more insulating layers 200 also do not cover certain edges of the conductive frame 102 (which are left exposed by the one or more insulating layers 200).

The implantable medical device 100 can also comprise one or more metallic components 104 affixed or otherwise coupled to the one or more insulating layers 200 (see, e.g., FIGS. 2A and 2B).

In certain embodiments, the implantable medical device 100 can be a stent electrode array and the conductive frame 102 can be the frame of the stent electrode array. In additional embodiments, the implantable medical device 100 can refer to any of stents, scaffolds, stent-electrode arrays, or electrode arrays disclosed in U.S. Patent Pub. No. 2020/0016396; U.S. Patent Pub. No. 2019/0336748; U.S. Patent Pub. No. US 2014/0288667; U.S. Pat. Nos. 11,883,671; 11,672,986; 11,550,391; 11,376,138; 11,093,038; 10,575,783; 10,485,968; 10,729,530, 10,512,555, the contents of which are incorporated herein by reference in their entireties.

In some embodiments, the conductive frame 102 can be made of a shape memory metallic alloy. For example, the conductive frame 102 can be made of a nickel-titanium alloy (e.g., Nitinol). Also, for example, at least part of the conductive frame 102 can be made of at least one of stainless steel, cobalt-chromium, gold, platinum, tungsten, aluminum, nickel-chromium alloy, gold-palladium-rhodium alloy, chromium-nickel-molybdenum alloy, iridium, rhodium, or a combination thereof. Alternatively, at least part of the conductive frame 102 can be made of a polymeric material (e.g., at least one of nylon, polyurethane, and polypropylene) covered by a conductive metallic layer.

In these and other embodiments, the one or more metallic components 104 can be made of a noble metal or noble metal alloy. For example, the the one or more metallic components 104 can be made of at least one of platinum, gold, and titanium.

A first conductive element 106 (e.g., a conductive trace, track, and/or wire) can connect the conductive frame 102 to a signal analyzer 108 (see, e.g., FIG. 1B). For example, the first conductive element 106 can be electrically coupled to at least part of the conductive frame 102 and can also be electrically coupled to the signal analyzer 108 via one or more lead wires of a sheathed lead wire bundle 110. As shown in FIG. 1A, the sheathed lead wire bundle 110 can comprise a proximal end 111. The proximal end 111 can be configured to be plugged into a part of the signal analyzer 108.

One or more second conductive elements 202 (e.g., conductive traces, tracks, and/or wires, see FIGS. 2A and 2B) can connect the one or more metallic components 104 to the signal analyzer 108 via additional lead wires of the sheathed lead wire bundle 110. The one or more lead wires can be insulated from the additional lead wires within the sheathed lead wire bundle 110.

In some embodiments, the first conductive element 106 and the one or more second conductive elements 202 can be made of at least one of gold, titanium, and platinum. For example, the first conductive element 106 or the one or more second conductive elements 202 can be implemented as gold, titanium, and/or platinum traces, tracks, and/or wires.

The signal analyzer 108 can be configured to determine a voltage signal within the subject using the conductive frame 102 and at least one of the metallic components 104 serving as electrodes. For example, the signal analyzer 108 can be configured to determine a voltage signal within the brain of the subject using the conductive frame 102 and at least one of the metallic components 104 serving as electrodes.

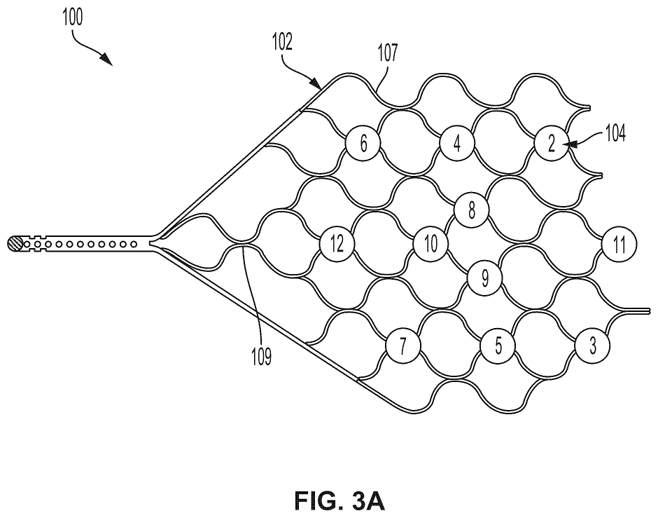

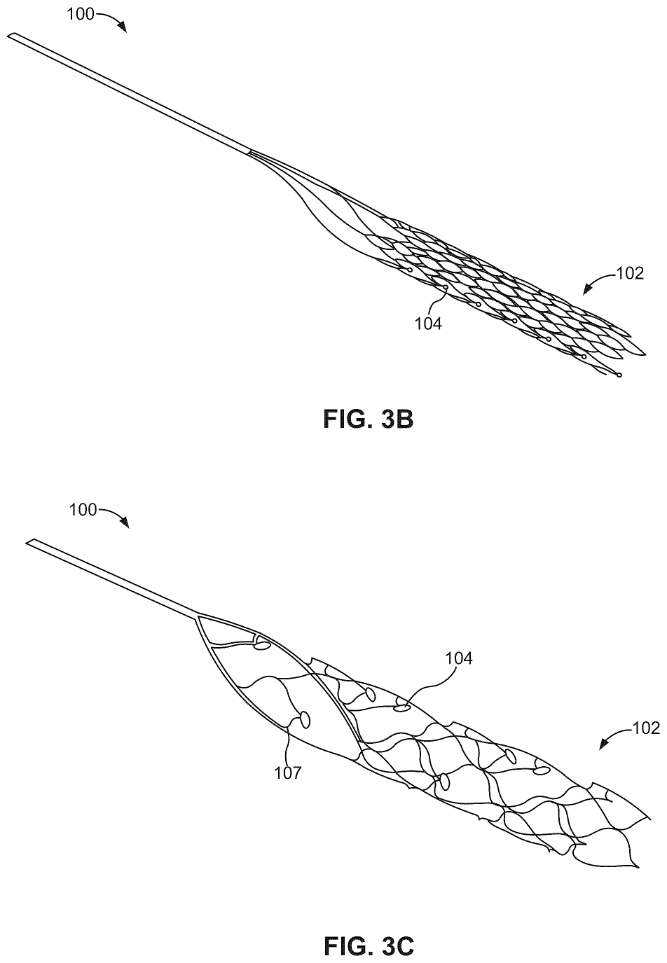

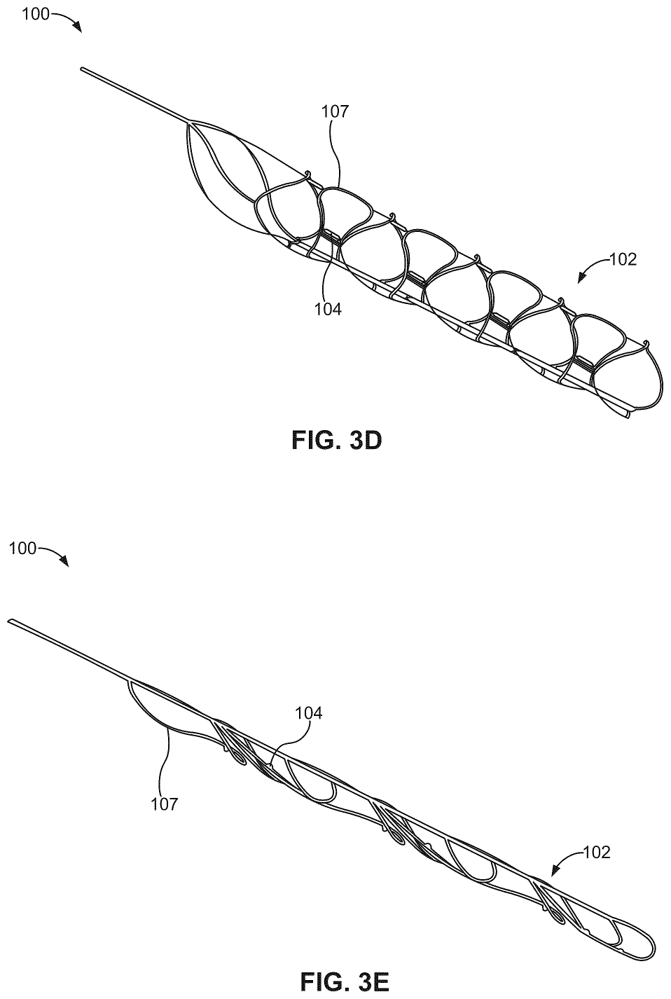

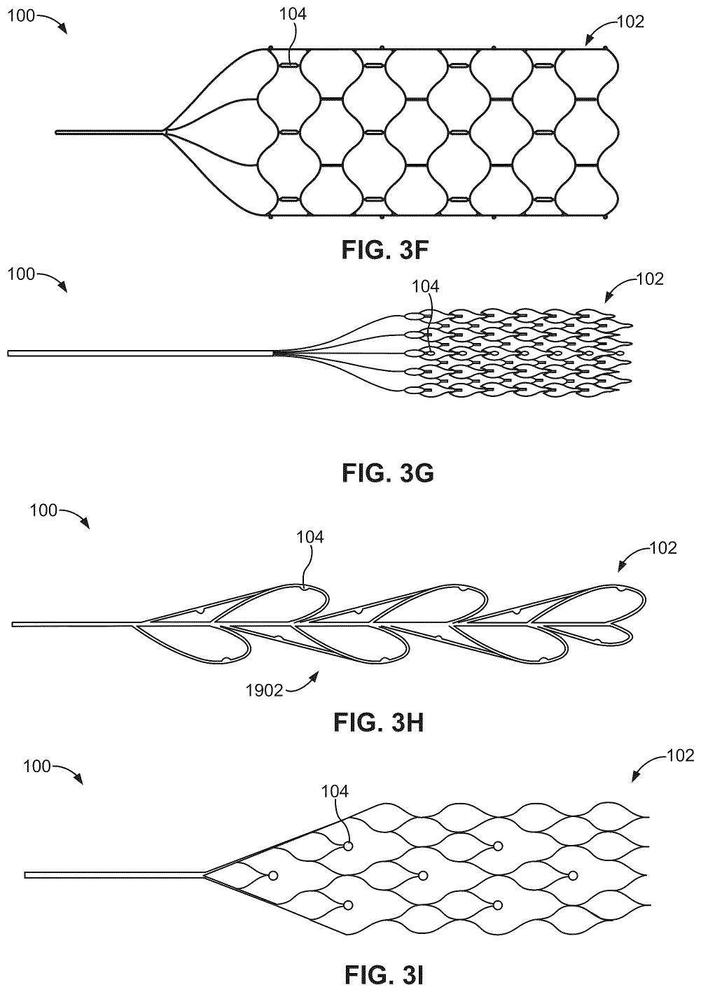

In some embodiments, the conductive frame 102 can be substantially tubular-shaped. In other embodiments, the conductive frame 102 can be substantially planar or flattened (see, e.g., FIGS. 3F-31).

The conductive frame can comprise a plurality of struts 107 connected by crosslinks 109. For example, each of the struts 107 can be a wire formed into at least one of a wave-like or undulating shape, a zig-zag shape, and a straight line.

In some embodiments, the conductive frame 102 can serve as the reference electrode and at least one of the metallic components 104 can serve as an active electrode or sensing/indicator electrode.

In these embodiments, the signal analyzer 108 can determine a voltage signal such as a local field potential, an event potential, or a neuronal action potential using the conductive frame 102 as the reference electrode and at least one of the metallic components 104 as the active electrode.

In some embodiments, the signal recordings or the processed signal recordings can be used to help subjects who are paralyzed due to neurological injury or disease to communicate or engage with the outside world by allowing the subject to directly control devices of software applications with their neural signals. Moreover, the signal recordings or the processed signal recordings can be used to improve a subject's mobility by allowing the subject to control mobility vehicles such as a wheelchair with the subject's neural signals.

In certain embodiments, the entirety of the conductive frame 102 can serve as the reference electrode while at least one of the metallic components 104 can serve as the active/indicator electrode. For example, the entirety of the conductive frame can have a total conductive frame surface area and the at least one metallic component 104 (or all of the metallic components 104 combined) can have a total component electrode surface area. A ratio of the total conductive frame surface area to the total component electrode surface area can be between about 3:1 and 30:1.

One technical advantage of using the entire conductive frame 102 as the reference electrode is that its larger surface area relative to the total surface area of the second electrodes 104 results in the conductive frame 102 having a lower impedance, thereby introducing less thermal noise. A reference electrode with a higher impedance can introduce an increased level of noise on all of the signals captured by the active electrodes.

In other embodiments, a portion of the conductive frame 102 can serve as the reference electrode. In these embodiments, the portion of the conductive frame 102 serving as the reference electrode can be between about 25% to about 95% of the conductive frame 102 by surface area. For example, the portion of the conductive frame 102 serving as the reference electrode can be between about 25% to about 95% of the total conductive frame surface area.

In some embodiments, the portion of the conductive frame 102 can be electrically insulated from another portion of the conductive frame 102 (e.g., by one or more insulators or insulating layer(s) 200) such that the conductive frame 102 is divided into multiple electrically conductive regions.

In alternative embodiments, the conductive frame 102 can serve as the active or indicator electrode and at least one of the metallic components 104 can serve as the reference electrode. In these embodiments, the signal analyzer 108 can determine a voltage signal such as a local field potential, an event potential, or a neuronal action potential using the conductive frame 102 as the active or indicator electrode and at least one of the metallic components 104 as the reference electrode.

In certain embodiments, the entirety of the conductive frame 102 can serve as the active electrode while at least one of the metallic components 104 can serve as the reference electrode. For example, the entirety of the conductive frame can have a total conductive frame surface area and the at least one metallic component 104 (or all of the metallic components 104 combined) can have a total component electrode surface area. A ratio of the total conductive frame surface area to the total component electrode surface area can be between about 3:1 and 30:1.

In other embodiments, a portion of the conductive frame 102 can serve as the active electrode. In these embodiments, the portion of the conductive frame 102 serving as the active electrode can be between 25% to 95% of the conductive frame 102 by surface area. In some embodiments, the portion of the conductive frame 102 can be electrically insulated from another portion of the conductive frame 102 by one or more insulators or insulating layer(s) such that the conductive frame 102 is divided into multiple electrically conductive regions.

FIG. 1B illustrates one embodiment of a system 112 for recording bio-signals and/or stimulating tissue or target(s) (e.g., neural tissue or neural target(s)) using the implantable medical device 100. In this sense, the system 112 can be a bio-signal recording system and/or a bio-stimulation system.

The system 112 can comprise the implantable medical device 100, the signal analyzer 108, and a computing device 114 communicatively coupled to the signal analyzer 108. The system 112 can also comprise one or more first conductive elements 106 and one or more second conductive elements 202.

In some embodiments, the signal analyzer 108 can be part of a telemetry unit 116. As will be discussed in more detail in later sections, the telemetry unit 116 can also comprise a pulse generator 118 or a pulse generating unit. The pulse generator 118 can be configured to generate an electrical impulse that is transmissible to the conductive frame 102 of the implantable medical device 100 to stimulate an intracorporeal target within the subject.

For example, the telemetry unit 116 (including the signal analyzer 108 and/or the pulse generator 118) can be implanted within a pectoral region or arm (e.g., forearm) of the subject.

In other components, the telemetry unit 116 (including the signal analyzer 108 and/or the pulse generator 118) can be an extracorporeal device or the telemetry unit 116 can be designed or configured such that least part of the telemetry unit 116 resides outside of the body of the subject.

FIG. 1B illustrates that the signal analyzer 108 can be electrically coupled or connected to the implantable medical device 100 via lead wires of a sheathed lead wire bundle 110. Each of the lead wires can also be electrically coupled or connected to the conductive frame 102 or a metallic component 104 of the implantable medical device 100.

As shown in FIG. 1B, the signal analyzer 108 can be wirelessly connected to a computing device 114 such as a laptop or desktop computing device. The computing device 114 can also be a tablet computer or a smartphone.

In certain embodiments, the computing device 114 is an extracorporeal computing device. In alternative embodiments, at least part of the computing device 114 can be implanted within the subject or the computing device 114 can refer to a processing component or processor unit of the implantable signal analyzer 108.

The signal analyzer 108 can be communicatively coupled to the extracorporeal computing device 114 via a wireless communication protocol or standard. For example, the signal analyzer 108 can be communicatively coupled to the extracorporeal computing device 114 via a 3G wireless communication standard, a 4G wireless communication standard, a 5G wireless communication standard, a long-term evolution (LTE) wireless communication standard, a Bluetooth™ (IEEE 802.15.1) or Bluetooth™ Lower Energy (BLE) short-range communication protocol, a wireless fidelity (WiFi) (IEEE 802.11) communication protocol, an ultra-wideband (UWB) (IEEE 802.15.3) communication protocol, a ZigBee™ (IEEE 802.15.4) communication protocol, or a combination thereof.

In some embodiments, the voltage signal measured by the signal analyzer 108 can be a local field potential (LFP), an event potential, or a neuronal action potential.

In certain embodiments, at least part of the signal analyzer 108 can be or act as a voltmeter. In these and other embodiments, the signal analyzer 108 can be part of the telemetry unit 116 and the telemetry unit 116 can also comprise an analog-to-digital converter and other electronic circuits. For example, the signal analyzer 108 of the telemetry unit 116 can digitize the voltage signal between the pairs of electrodes (the active electrode and the reference electrode) and then transmit the digitized voltage signal to the extracorporeal computing device 114 to analyze the signal, calculate a power spectral density, or perform additional signal processing based on the LFP, the event potential, or the neuronal action potential.

In some embodiments, the telemetry unit 116 can comprise a conductive case 120. For example, the conductive case 120 can be made of titanium. Also, for example, the conductive case 120 can be made of stainless steel, gold, or platinum.

In certain embodiments, the conductive case 120 of the telemetry unit 116 can serve as the reference electrode and either the conductive frame 102 or one or more of the metallic components 104 can serve as the active electrode.

As previously discussed, in some embodiments, the system 112 can also be used to stimulate tissue or target(s) (e.g., neural tissue or neural target(s)) within the subject using the implantable medical device 100.

In these embodiments, the system 112 can comprise a conductive frame 102 and a pulse generator 118 electrically coupled to the conductive frame 102. The conductive frame 102 can be implanted near an intracorporeal target within the subject. The pulse generator 118 can be configured to generate an electrical impulse that is transmissible to the conductive frame 102 to stimulate the intracorporeal target.

In some embodiments, the conductive frame 102 can be configured to be implanted within the brain of the subject (e.g., within cortical vessels). The conductive frame 102 can also be configured to be implanted at locations along the surface of the brain, at locations exterior to brain vessels, or within the dura mater of the subject.

In some embodiments, the entirety of the conductive frame 102 can be used to stimulate the intracorporeal target.

In other embodiments, a portion of the conductive frame 102 can be used to stimulate the intracorporeal target. For example, the portion of the conductive frame 102 can be between about 25% to about 95% of the entirety of the conductive frame 102 by surface area.

In some embodiments, generating the electrical impulse can further comprise generating the electrical impulse by increasing a current amplitude of the electrical impulse from 0 mA to up to 10 mA in 0.1 mA steps. Moreover, generating the electrical impulse can further comprise increasing a voltage of the electrical impulse from 0 V to up to 10 V in 0.25 V steps. In addition, a pulse width of the electrical impulse generated can be configured to be between about 25 uS to about 600 uS. Furthermore, a frequency of the electrical impulse generated can be configured to be between 0.5 Hz and 10,000 Hz.

FIG. 2A is a schematic representation of a cross-section of part of the implantable medical device 100. As shown in FIG. 2A, one or more insulating layers 200 can be disposed on at least part of the conductive frame 102. In some embodiments, the one or more insulating layers 200 can cover a radially outward-facing surface or upper/top surface of the conductive frame 102. In these embodiments, the one or more insulating layers 200 do not cover an inner surface, a radially inward-facing surface, or a bottom surface of the conductive frame 102 (which are left exposed by the one or more insulating layers 200). In certain embodiments, the one or more insulating layers 200 also do not cover certain edges of the conductive frame 102 (which are left exposed by the one or more insulating layers 200).

As previously discussed, the conductive frame 102 can be made of a shape memory metallic alloy. For example, the shape memory metallic alloy can be a nickel-titanium alloy (Nitinol). As a more specific example, the conductive frame 102 can be a lattice structure made of a thin film of the shape memory metallic alloy (e.g., Nitinol).

In other embodiments, the conductive frame 102 can be made in part of at least one of stainless steel, cobalt-chromium, and magnesium. In alternative embodiments, at least part of the conductive frame 102 can be made of a polymeric material (e.g., at least one of nylon, polyurethane, and polypropylene) covered by a conductive metallic layer.

In some embodiment, the overall shape of the conductive frame 104 can be substantially tubular-shaped or shaped like a rolled-up lattice-structure (see, e.g., FIG. 3B). In other embodiments, the conductive frame can be substantially planar or flattened (see, e.g., FIGS. 3F-31).

At least one of the insulating layers 200 can be made of a non-conductive material such as a non-conductive ceramic. For example, at least one of the insulating layers 200 can be made of yttria stabilized zirconia (YSZ). In other embodiments, at least one of the insulating layers 200 can be made of silicon dioxide.

As shown in FIG. 2A, the implantable medical device 100 can comprise a first insulating layer 200A and a second insulating layer 200B. The first insulating layer 200A can be disposed on or cover at least part of the conductive frame 102. For example, the first insulating layer 200A can be disposed on or cover a radially outward-facing surface or upper/top surface of the conductive frame 102. The second insulating layer 200B can be disposed on or cover at least part of the first insulating layer 200A.

As shown in FIG. 2A, in one embodiment, both the first insulating layer 200A and the second insulating layer 200B can be made of YSZ.

The second insulating layer 200B can secure or affix the metallic components 104 to the remainder of the implantable medical device 100. For example, the second insulating layer 200B can secure or affix the metallic components 104 to the first insulating layer 200A covering the conductive frame 102. As shown in FIG. 2A, the second insulating layer 200B can expose part of the metallic component 104.

The first insulating layer 200A can be shaped or designed to accommodate each of the metallic components 104. For example, an outer or top-most surface of the first insulating layer 200A can be shaped or configured such that a plurality of divots or cavities are defined along the outer or top-most surface of the first insulating layer 200A. Each of the metallic components 104 can be placed or positioned within the divot or cavity defined along the outer or top-most surface of the first insulating layer 200A. Once each of the metallic components 104 is placed or positioned within a divot or cavity defined along the outer or top-most surface of the first insulating layer 200A, a second insulating layer 200B can cover at least part of the metallic components 104 to affix or secure the metallic components 104 to the first insulating layer 200A covering the conductive frame 102. The second insulating layer 200B can be shaped or configured to have raised or angled edges to secure the metallic component 104 to the first insulating layer 200A.

As previously discussed, in some embodiments, at least one of the metallic components 104 can be made of a noble metal or noble metal alloy. For example, at least one of the metallic components 104 can be made of platinum or platinum black, gold, or titanium.

In some embodiments, the metallic components 104 can be shaped as circular disks. In other embodiments, the metallic components 104 can be shaped as elliptical disks or oblong disks.

As shown in FIG. 2A, a second conductive element 202 can electrically couple or connect each of the metallic components 104 to a signal analyzer 108. The second conductive element 202 can be a conductive trace, track or wire extending through the insulating layers 200. For example, the conductive elements 202 can be conductive traces, tracks, or wires made of gold, titanium, or platinum. As a more specific example, the second conductive elements 202 can be conductive gold, titanium, or platinum traces, tracks, or wires extending through the first insulating layers 200A, the second insulating layer 200B, or a combination thereof.

In some embodiments, a first conductive element 106 (see, e.g., FIG. 1A) can also extend through one or more insulating layers 200. The first conductive element 106 can be a conductive trace, track, or wire extending through one or more insulating layers 200. For example, the first conductive element 106 can be a conductive trace, track, or wire made of gold, titanium, or platinum. Although not shown in FIG. 2A, the first conductive element 106 can extend through at least part of the first insulating layer 200A. In other embodiments, the first conductive element 106 can be directly coupled to a proximal end of the conductive frame 102. The first conductive element 106 can electrically couple or connect the conductive frame 102 to the signal analyzer 108.

As previously discussed, the signal analyzer can be configured to determine a voltage signal within the subject using the conductive frame 102 as a first electrode and at least one of the metallic components 104 as a second electrode.

In some embodiments, the entirety of the conductive frame 102 can serve as an electrode such as a reference electrode or an active/indicator electrode.

In other embodiments, a portion of the conductive frame 102 can serve as an electrode such as a reference electrode or an active/indicator electrode. For example, the portion of the conductive frame can be between about 25% to about 95% of the entirety of the conductive frame 102 by surface area. In these embodiments, the portion of the conductive frame 102 can be electrically insulated from another portion of the conductive frame 102 by one or more of the insulating layers 200 such that the conductive frame 102 is divided into multiple electrically conductive regions.

FIG. 2B illustrates a close-up partially exploded view of an electrode mounting site 204 of the implantable medical device 100. In some embodiments, the electrode being mounted can be one of the metallic components 104.

In some embodiments, the conductive frame 104 can comprise a plurality of struts 107 connected by crosslinks 109. Each of the struts 107 can be a wire or strand formed into a wave-like or undulating shape, a zig-zag shape, a straight line, or a combination thereof. Each of the metallic components 104 can be positioned at a linkage/meeting point between adjacent struts 107 or positioned at or near a crosslink 109 connecting adjacent struts 107.

As shown in FIG. 2B, the struts 107 and crosslinks 109 of the conductive frame 102 can be covered by a first insulating layer 200A. A metallic component 104 can be positioned or otherwise placed at a linkage point or meeting point between adjacent struts 107 or along a crosslink 109 connecting adjacent struts 107. A second conductive element 202 (e.g., a metallic trace or wire) can be electrically coupled or connected to the metallic component 104.

In some embodiments, the second conductive element 202 can follow a path along one or more struts 107 and crosslinks 109 of the conductive frame 102 until the second conductive element 202 connects with one of the lead wires of the sheathed lead wire bundle 110.

A second insulating layer 200B that substantially matches the shape of the first insulating layer 200A can then cover the first insulating layer 200A. The second insulating layer 200B can be shaped or designed to have apertures or windows 206 that expose at least part of each of the metallic components 104. The second insulating layer 200B can cover the second conductive element 202.

In some embodiments, the same insulating layer (e.g., the second insulating layer 200B) can cover multiple conductive elements (e.g., multiple instances of the second conductive element 202). For example, each of the conductive elements can be a conductive trace having a trace width. As a more specific example, the trace width of each of the conductive traces can be about 30 μm. A distance separating the conductive traces can be between about 10 μm and 30 μm.

Although FIG. 2B only depicts a first insulating layer 200A and a second insulating layer 200B, it is contemplated by this disclosure that the implantable medical device 100 can comprise one or more additional insulating layers 200 including a third insulating layer, a fourth insulating layer, a fifth insulating layer, etc. Each of the insulating layers 200 can insulate or separate a conductive element (e.g., a conductive trace, track, wire, etc.) electrically coupled or connected to a metallic component 104 from another conductive element electrically coupled or connected to another metallic component 104.

As previously discussed, each of the metallic components 104 can serve as an electrode of the implantable medical device 100. For example, each of the metallic components 104 can serve as an active electrode or an indicator electrode.

In alternative embodiments, at least one of the metallic components 104 can serve as a reference electrode.

One technical problem faced by the applicant is how to increase the number of electrodes of the implantable medical device 100 without decreasing the width of the conductive traces connecting the electrodes to the lead wires. Decreasing the width of a conductive trace may result in an increase in the resistance of the conductive trace, thereby increasing noise interference. One technical solution discovered and developed by the applicant is the implantable medical device 100 disclosed herein with a plurality of stacked insulating layers 200 that allow the implantable medical device 100 to carry more electrodes.

FIG. 3A illustrates another embodiment of the implantable medical device 100 comprising a conductive frame 102 and a plurality of metallic components 104 affixed to various locations along the conductive frame 102. Each of the metallic components 104 can serve as an electrode of the implantable medical device 100.

In some embodiments, one or more of the metallic components 104 can serve as an active electrode while the entire conductive frame 102 serves as a reference electrode for recording neural signals within a subject.

One technical advantage of using the conductive frame 102 as the reference electrode is that the conductive frame 102, since it also acts as the base substrate for the metallic components 104, is collocated with the metallic components 104 and faces all of the same directions as the metallic components 104. Therefore, any noise picked up by the conductive frame 102 is more closely correlated with the noise picked up by the metallic components 104 serving as the active electrodes, which makes for a more useful reference subtraction.

In alternative embodiments, at least one of the metallic components 104 can serve as a reference electrode while the entire conductive frame 102 serves as an active electrode for recording neural signals within a subject.

In some embodiments, the conductive frame 102 can be collapsible, foldable, or deformable. In other embodiments, the conductive frame 102 can be flattened or substantially planar-shaped (see FIGS. 3F-31).

As shown in FIG. 3A, in some embodiments, the implantable medical device 100 can comprise eleven metallic components 104 serving as electrodes. In other embodiments, the implantable medical device 100 can comprise between two metallic components 104 and ten metallic components 104. In further embodiments, the implantable medical device 100 can comprise between twelve metallic components 104 and up to twenty metallic components 104.

In some embodiments, each of the metallic components 104 can be positioned at a linkage point or meeting point between adjacent struts 107. In other embodiments, at least one of the second electrodes 104 can be positioned along one of the struts 107 not at a linkage point or meeting point between adjacent struts 107.

The conductive frame 102 can have a total conductive frame surface area and the plurality of metallic components 104 can have a total component electrode surface area. In some embodiments, a ratio of the total conductive frame surface area to the total component electrode surface area can be between about 3:1 and 30:1.

In some embodiments, the conductive frame 102 can have a total conductive frame surface area of about 60 mm². In these embodiments, each of the metallic components 104 can have an electrode area of about 0.433 mm². When the implantable medical device 100 comprises eleven metallic components 104, the total component electrode surface area can be about 4.77 mm².

FIGS. 3B-3E illustrate additional embodiments of the implantable medical device 100 comprising conductive frames 102 of various shapes and configurations. The medical devices 100 shown in FIGS. 3B-3E can operate similar to the medical device 100 shown in FIG. 1A.

For example, FIG. 3B illustrates an embodiment of the implantable medical device 100 comprising a substantially tubular-shaped conductive frame 102 comprising struts 107 forming a plurality of piriform structures and metallic components 104 coupled to the ends or tips of the piriform structures. The metallic components 104 can be aligned in a linear fashion or aligned longitudinally (e.g., in a row) with respect to the tubular frame body.

FIG. 3C illustrates an embodiment of the implantable medical device 100 comprising a substantially tubular stent body comprising struts 107 forming a plurality of deformed or collapsed limoniform structures and metallic components 104 serving as tips or ends of the deformed/collapsed limoniform structures.

FIG. 3D illustrates another embodiment of the implantable medical device 100 comprising a substantially tubular stent body comprising struts 107 forming alternating leaf-like wire structures and metallic components 104 serving as connectors connecting the leaf-like wire structures.

FIG. 3E illustrates yet another embodiment of the implantable medical device 100 comprising struts 107 forming collapsed hoops and metallic components 104 coupled to the collapsed hoops.

FIGS. 3F-31 illustrate alternative embodiments of the implantable medical device 100 comprising flattened or planar-shaped conductive frames 102. Such flattened or planar-shaped conductive frames 102 can be similar to the tubular-shaped conductive frames 102 shown in FIGS. 3B-3E except such flattened or planar-shaped designs allow the conductive frames 102 to lie flat against certain surfaces of the brain, within the dura mater of the subject, or within the subarachnoid space.

The conductive frame 102 of the implantable medical device 100 can also be similar in shape to any of the frames disclosed in U.S. Pat. Nos. 10,575,783; 10,485,968, or 10,729,530, the contents of which are incorporated herein by reference in their entireties.

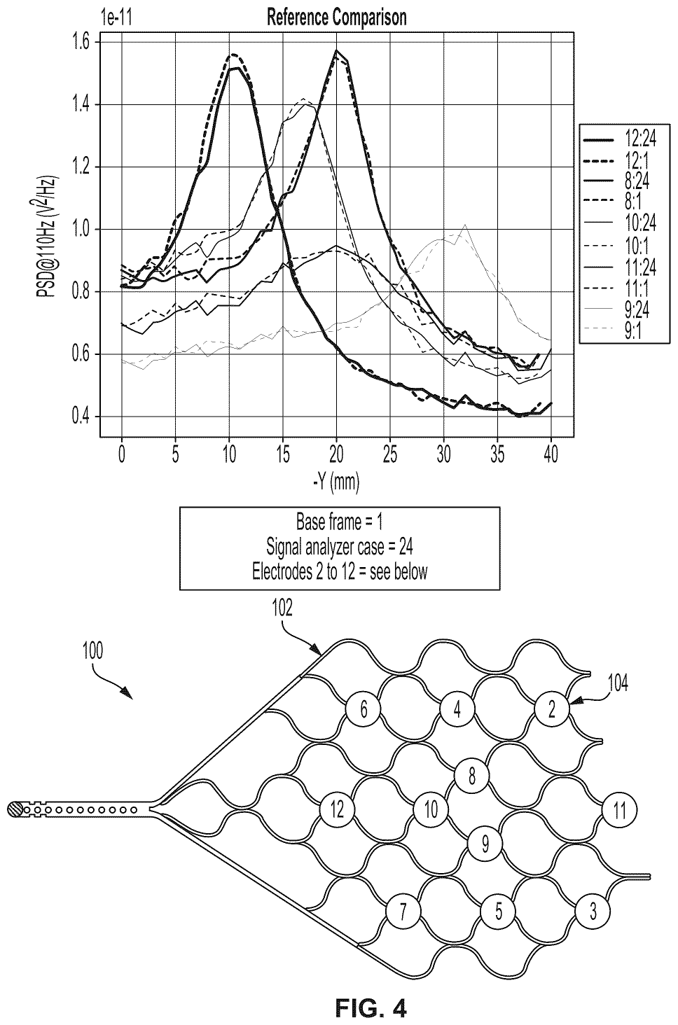

FIG. 4 is a graph comparing the use of the conductive frame 102 as a reference electrode against using the conductive case 120 of the signal analyzer 108 as the reference electrode (see, e.g., FIG. 1B).

As shown in FIG. 4, the amplitude and spatial sensitivity of signal recordings made using the conductive frame 102 as the reference electrode (shown on the graph using 12:1, 8:1, 10:1, 11:1, and 9:1) were equivalent to the signal recordings made using the conductive case 120 (e.g., the titanium case) (shown on the graph using the number “24”) of the signal analyzer 108 (see, e.g., FIG. 1B) as the reference electrode (shown on the graph using 12:24, 8:24, 10:24, 11:24, and 9:24). The number “1” on the graph refers to the conductive frame 102, the number “24” on the graph refers to the conductive case 120 of the signal analyzer 300, and the other numbers refer to the numbered metallic components 104 of the implantable medical device 100 (see bottom illustration of FIG. 4). For purposes of this comparison, the numbered metallic components 104 serve as active electrodes.

The y-axis of the graph shows the recorded signal expressed as the power spectral density (PSD) in V²/Hz. The x-axis of the graph shows the positions of the metallic components 104 along the longitudinal axis or length of the implantable medical device 100. The numbers are multiplied by −1 (hence the −Y) to align the data with the illustration of the implantable medical device 100 below the graph.

However, as previously discussed, since the conductive frame 102 also acts as the base substrate for the metallic components 104, the conductive frame 102 is essentially collocated with the metallic components 104 and faces all of the same directions as the metallic components 104. Therefore, any noise picked up by the conductive frame 102 while serving as the reference electrode is more closely correlated with the noise picked up by the metallic components 104 serving as the active electrodes.

FIG. 5 are graphs comparing the use of the conductive frame 102 as a reference electrode and use of various metallic components 104 as the reference electrode.

As shown in FIG. 5, when the conductive frame 102 was used as the reference electrode (shown on the graph on the left as 12:1, 10:1, and 11:1), the signal recordings yielded larger amplitudes than when each of the various metallic components 104 was used as the reference electrode (shown on both graphs as 12:7, 12:5, 12:3, 10:7, 10:5, 10:3, 11:7, 11:5, and 11: 3). The difference in amplitude was likely caused by the larger voltage gradient difference when the conductive frame 102 was used as the reference electrode compared to using pairs of the metallic components 104 as both the active and the reference electrodes.

FIG. 6 illustrates certain veins and sinuses of the subject that can serve as implantation sites for the medical device 100 disclosed herein.

In some embodiments, the medical device 100 can be implanted within a venous sinus of the subject. For example, the medical device 100 can be implanted within a superior sagittal sinus 600, an inferior sagittal sinus 602, a sigmoid sinus 604, a transverse sinus 606, or a straight sinus 608.

In other embodiments, the medical device 100 can be implanted within a superficial cerebral vein of the subject. For example, the medical device 100 can be implanted within at least one of a vein of Labbe 610, a vein of Trolard 612, a Sylvian vein 614, and a Rolandic vein 616.

The medical device 100 can also be implanted within a deep cerebral vein of the subject. For example, the medical device 100 can be implanted within at least one of a vein of Rosenthal 618, a vein of Galen 620, a superior thalamostriate vein 622, an inferior thalamostriate vein, and an internal cerebral vein 624.

In further embodiments, the medical device 100 can also be implanted within at least one of a central sulcal vein, a post-central sulcal vein, and a pre-central sulcal vein. In additional embodiments, the medical device 100 can also be implanted or configured to be implanted within a vessel extending through a hippocampus or amygdala of the subject.

Once implanted, the medical device 100 can be configured to detect or record an electrophysiological signal of the subject and/or stimulate an intracorporeal target within the subject. In some embodiments, the electrophysiological signal can be a raw neural signal, transient oscillatory or pseudo-oscillatory bursts or burst features, a binarized neural signal, an action potential, an event-related potential, a graded potential, a field potential, a rhythmic or repetitive pattern of neural activity, chunks or sequences of any of the foregoing, or combinations of any of the foregoing. For example, the electrophysiological signal can be a local field potential (LFP) or an intracranial/cortical EEG measured within a cerebral or cortical vessel (e.g., a venous sinus or cortical vein). Also, for example, the electrophysiological signal can be an electrocorticography (ECoG) signal.

As previously discussed, when the intracorporeal target to be stimulated by the medical device 100 is a vagus nerve of the subject, the medical device 100 can be implanted within an internal jugular vein (either a right internal jugular vein 626 or a left internal jugular vein 628) or an internal carotid artery.

In other embodiments, the intracorporeal target or stimulation target can be the cerebellum 630 of the subject. In these embodiments, the medical device 100 can be implanted within at least one of a sigmoid sinus 604 and a straight sinus 608 of the subject. Moreover, the medical device 100 can also be implanted within a transverse sinus 606 of the subject. At least part of the cerebellum 630 is adjacent to the sigmoid sinus 604, the straight sinus 608, and the transverse sinus 606.

In additional embodiments, the medical device 100 can also record neural signals and/or stimulate neural tissue at locations along the surface of the brain, at locations exterior to brain vessels, or within the dura mater of the subject.

In some embodiments, stimulating the intracorporeal target or the stimulation target via the medical device 100 can increase blood flow to the intracorporeal target or raise levels of certain neurotransmitters involved in suppressing seizure activity. Moreover, stimulating the intracorporeal target via the medical device 100 can also lead to sodium-channel inactivation (using high-frequency stimulation), long-term depression of certain neurotransmitters (using high-frequency stimulation), and/or glutamatergic depression (using both low-frequency and high-frequency stimulation).

For example, when stimulating cortical or cerebral targets, the electrical impulse can be bipolar with the voltage of the electrical impulse increased from 1V to 7 V in 0.25 V steps. The electrical impulse generated can have a pulse width of between 90 uS to about 540 uS, a frequency between about 3 Hz to 5 Hz in a low-frequency range, and a frequency between about 50 Hz to 130 Hz in a high-frequency range.

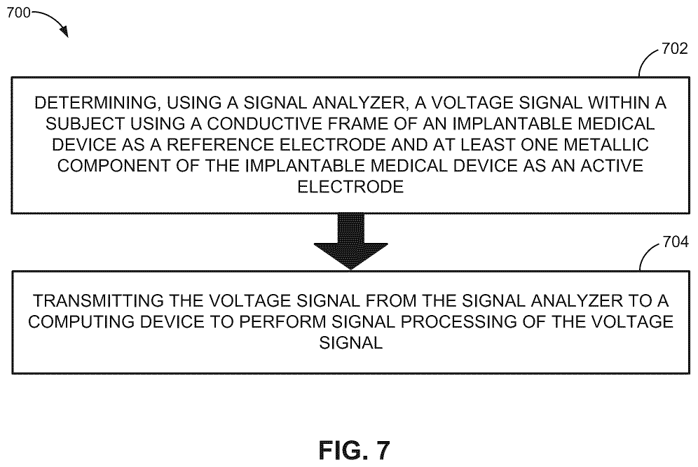

FIG. 7 illustrates a method 700 of analyzing a bio-signal obtained from within a body of a subject. The method 700 can comprise determining or capturing, using one or more processors of a signal analyzer 108, a voltage signal within a subject using a conductive frame 102 of an implantable medical device 100 as a reference electrode and at least one metallic component 104 of the implantable medical device 100 as an active electrode in step 702.

The implantable medical device 100 can further comprise one or more insulating layers 200 disposed on at least part of the conductive frame 102. In some embodiments, the one or more insulating layers 200 can cover a radially outward-facing surface or upper/top surface of the conductive frame 102. In these embodiments, the one or more insulating layers 200 do not cover an inner surface, a radially inward-facing surface, or a bottom surface of the conductive frame 102 (which are left exposed by the one or more insulating layers 200). In certain embodiments, the one or more insulating layers 200 also do not cover certain edges of the conductive frame 102 (which are left exposed by the one or more insulating layers 200).

The at least one metallic component 104 can be affixed to the one or more insulating layers 200. The conductive frame 102 can be coupled to the signal analyzer 108 via one or more first conductive elements 106 and the at least one metallic component 104 can be coupled to the signal analyzer 108 via one or more second conductive elements 202.

The method 700 can also comprise transmitting the voltage signal from the signal analyzer to a computing device 114 to perform signal processing of the voltage signal in step 704.

In some embodiments, the entirety of the conductive frame 102 can serve as the reference electrode. In these embodiments, the entirety of the conductive frame 102 can have a total conductive frame surface area. The at least one metallic component 104 can have a total component electrode surface area. A ratio of the total conductive frame surface area to the total component electrode surface area can be between about 3:1 and 30:1.

In alternative embodiments, a portion of the conductive frame 102 can serve as the reference electrode. In these embodiments, the portion of the conductive frame 102 can be between 25% to 95% of the entirety of the conductive frame 102 by surface area.

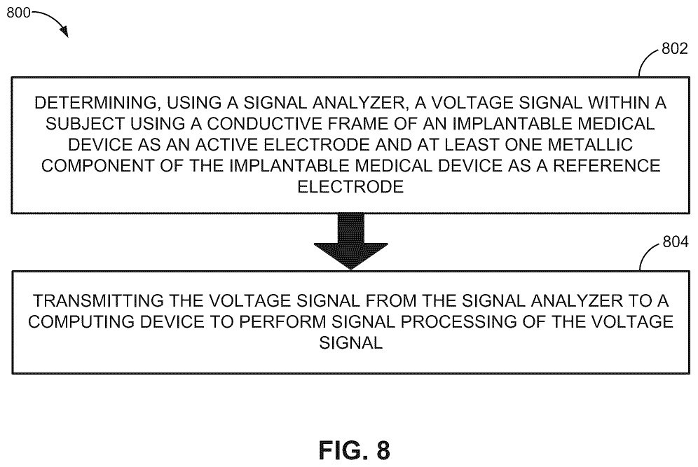

FIG. 8 illustrates another method 800 of analyzing a bio-signal obtained from within a body of a subject. The method 800 can comprise determining or capturing, using one or more processors of a signal analyzer 108, a voltage signal within a subject using a conductive frame 102 of an implantable medical device 100 as an active electrode and at least one metallic component 104 of the implantable medical device 100 as a reference electrode in step 802.

The implantable medical device 100 can further comprise one or more insulating layers 200 disposed on at least part of the conductive frame 102. In some embodiments, the one or more insulating layers 200 can cover a radially outward-facing surface or upper/top surface of the conductive frame 102. In these embodiments, the one or more insulating layers 200 do not cover an inner surface, a radially inward-facing surface, or a bottom surface of the conductive frame 102 (which are left exposed by the one or more insulating layers 200). In certain embodiments, the one or more insulating layers 200 also do not cover certain edges of the conductive frame 102 (which are left exposed by the one or more insulating layers 200).

The at least one metallic component 104 can be affixed to the one or more insulating layers 200. The conductive frame 102 can be coupled to the signal analyzer 108 via one or more first conductive elements 106 and the at least one metallic component 104 can be coupled to the signal analyzer 108 via one or more second conductive elements 202.

The method 800 can also comprise transmitting the voltage signal from the signal analyzer to a computing device 114 to perform signal processing of the voltage signal in step 804.

In some embodiments, the entirety of the conductive frame 102 can serve as the active electrode. In these embodiments, the entirety of the conductive frame 102 can have a total conductive frame surface area. The at least one metallic component 104 can have a total component electrode surface area. A ratio of the total conductive frame surface area to the total component electrode surface area can be between about 3:1 and 30:1.

In alternative embodiments, a portion of the conductive frame 102 can serve as the active electrode. In these embodiments, the portion of the conductive frame 102 can be between 25% to 95% of the entirety of the conductive frame 102 by surface area.

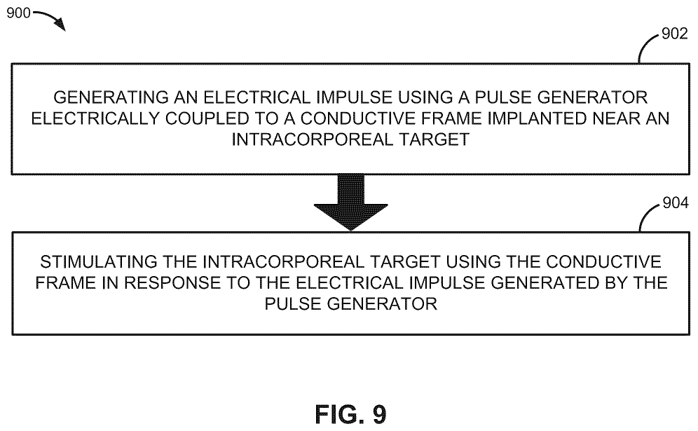

FIG. 9 illustrates a method 900 of stimulating an intracorporeal target. The method 900 can comprise generating an electrical impulse using a pulse generator 118 electrically coupled to a conductive frame 102 implanted near an intracorporeal target in operation 902. The method 900 can also comprise stimulating the intracorporeal target using the conductive frame 102 in response to the electrical impulse generated by the pulse generator 118 in operation 904.

The conductive frame 102 can be configured to be implanted within the brain of the subject, at locations along the surface of the brain, at locations exterior to brain vessels, or within the dura mater of the subject.

In some embodiments, the entirety of the conductive frame 102 can be used to stimulate the intracorporeal target.

In alternative embodiments, a portion of the conductive frame 102 can be used to stimulate the intracorporeal target. In these embodiments, the portion of the conductive frame 102 can be between 25% to 95% of the entirety of the conductive frame 102 by surface area.

In some embodiments, the conductive frame 102 can be substantially tubular-shaped.

In other embodiments, the conductive frame 102 can be substantially planar or flattened.

The conductive frame 102 can comprise a plurality of struts connected by crosslinks. Each of the struts can be a wire formed into at least one of a wave-like or undulating shape, a zig-zag shape, and a straight line.

The conductive frame 102 can also be made of a shape memory metallic alloy. For example, the conductive frame 102 can be made of a nickel-titanium alloy (e.g., Nitinol). In some embodiments, the pulse generator can be implantable.

The pulse generator 118 can generate the electrical impulse by increasing a current amplitude of the electrical impulse from 0 mA to up to 10 mA in 0.1 mA steps. The pulse generator 118 can also generate the electrical impulse by increasing a voltage of the electrical impulse from 0 V to up to 10 V in 0.25 V steps.

In some embodiments, a pulse width of the electrical impulse generated can be between about 25 uS to about 600 uS. A frequency of the electrical impulse generated can be between 0.5 Hz and 10,000 Hz.

One technical advantage of using the conductive frame 102 (e.g., the entire conductive frame 102) for stimulation is that given the larger surface area of the conductive frame 102, the conductive frame 102 would have a lower charge density for every stimulation amplitude and pulse width under the same stimulation frequency when compared to using any of the small metallic components 104 as stimulation electrodes.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit, or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of” when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including,” “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially,” “about,” and “approximately” as used herein mean the specified value or the specified value and a reasonable amount of deviation from the specified value (e.g., a deviation of up to +0.1%, +1%, +5%, or +10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

It will be understood by one of ordinary skill in the art that the various methods disclosed herein may be embodied in a non-transitory readable medium, machine-readable medium, and/or a machine accessible medium comprising instructions compatible, readable, and/or executable by a processor or server processor of a machine, device, or computing device. The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. An implantable medical device, comprising:
a conductive frame;
one or more insulating layers disposed on at least part of the conductive frame;
at least one metallic component affixed to the one or more insulating layers;
a first conductive element electrically coupled to at least part of the conductive frame; and
a second conductive element electrically coupled to the at least one metallic component,
wherein the first conductive element and the second conductive element are configured to be electrically coupled to a signal analyzer configured to determine a voltage signal within a subject using the conductive frame as a reference electrode and the at least one metallic component as an active electrode.

2. The implantable medical device of claim 1, wherein an entirety of the conductive frame serves as the reference electrode.

3. The implantable medical device of claim 2, wherein the entirety of the conductive frame has a total conductive frame surface area, wherein the at least one metallic component has a total component electrode surface area, and wherein a ratio of the total conductive frame surface area to the total component electrode surface area is between about 3:1 and 30:1.

4. The implantable medical device of claim 1, wherein a portion of the conductive frame serves as the reference electrode.

5. The implantable medical device of claim 4, wherein the portion of the conductive frame is between 25% to 95% of an entirety of the conductive frame by surface area.

6. The implantable medical device of claim 4, wherein the portion of the conductive frame is electrically insulated from another portion of the conductive frame such that the conductive frame is divided into multiple electrically conductive regions.

7. The implantable medical device of claim 1, wherein the conductive frame is substantially tubular-shaped.

8. The implantable medical device of claim 1, wherein the conductive frame is substantially planar or flattened.

9. The implantable medical device of claim 1, wherein the conductive frame comprises a plurality of struts connected by crosslinks.

10. The implantable medical device of claim 9, wherein each of the struts is a wire formed into at least one of a wave-like or undulating shape, a zig-zag shape, and a straight line.

11. The implantable medical device of claim 9, wherein the at least one metallic component is positioned at a linkage point or meeting point between adjacent struts.

12. The implantable medical device of claim 1, wherein the signal analyzer is part of a telemetry unit.

13. The implantable medical device of claim 1, wherein the signal analyzer is implantable.

14. The implantable medical device of claim 1, wherein the signal analyzer is an extracorporeal device.

15. The implantable medical device of claim 1, wherein the voltage signal is at least one of a field potential, an event potential, and a neuronal action potential.

16. The implantable medical device of claim 1, wherein the conductive frame is made of a shape memory metallic alloy.

17. The implantable medical device of claim 1, wherein the first conductive element and the second conductive element are conductive traces embedded within the one or more insulating layers.

18. The implantable medical device of claim 1, wherein the at least one metallic component is made of a noble metal or noble metal alloy.

19. The implantable medical device of claim 18, wherein the at least one metallic component is made of at least one of gold, titanium, and platinum.

20. The implantable medical device of claim 1, wherein at least one of the insulating layers is made of yttria stabilized zirconia or silicon dioxide.

21. A method of analyzing a signal, comprising:
determining, using one or more processors of a signal analyzer, a voltage signal within a subject using a conductive frame of an implantable medical device as a reference electrode and at least one metallic component of the implantable medical device as an active electrode,
wherein the implantable medical device further comprises:
one or more insulating layers disposed on at least part of the conductive frame, wherein the at least one metallic component is affixed to the one or more insulating layers,
a first conductive element electrically connecting at least part of the conductive frame to the signal analyzer, and
a second conductive element electrically connecting the at least one metallic component to the signal analyzer.

22-40. (canceled)

41. An implantable medical device, comprising:
a conductive frame;
one or more insulating layers disposed on at least part of the conductive frame;
at least one metallic component affixed to the one or more insulating layers;
a first conductive element electrically coupled to at least part of the conductive frame; and
a second conductive element electrically coupled to the at least one metallic component,
wherein the first conductive element and the second conductive element are configured to be electrically coupled to a signal analyzer configured to determine a voltage signal within a subject using the conductive frame as an active electrode and the at least one metallic component as a reference electrode.

42-60. (canceled)

61. A method of analyzing a signal, comprising:
determining, using one or more processors of a signal analyzer, a voltage signal within a subject using a conductive frame of an implantable medical device as an active electrode and at least one metallic component of the implantable medical device as a reference electrode,
wherein the implantable medical device further comprises:
one or more insulating layers disposed on at least part of the conductive frame, wherein the at least one metallic component is affixed to the one or more insulating layers,
a first conductive element electrically connecting at least part of the conductive frame to the signal analyzer, and
a second conductive element electrically connecting the at least one metallic component to the signal analyzer.

62-80. (canceled)

81. An implantable stimulation system, comprising:
a conductive frame, wherein the conductive frame is implantable near an intracorporeal target within a subject; and
a pulse generator electrically coupled to the implantable conductive frame, wherein the pulse generator is configured to generate an electrical impulse that is transmissible to the implantable conductive frame to stimulate the intracorporeal target.

82-90. (canceled)

91. A method of stimulating an intracorporeal target, comprising:
generating an electrical impulse using a pulse generator electrically coupled to a conductive frame implanted near an intracorporeal target; and
stimulating the intracorporeal target using the conductive frame in response to the electrical impulse generated by the pulse generator.

92-105. (canceled)
